(19) United States
(12) Patent Application Publication
(10) Pub. No.: US 2002/0009793 A1
(43) Pub. Date: Jan. 24, 2002

(54) Method of and apparatus for purifying an exhaust gas

(75) Inventor: Emil A.J. Wieser-Linhart, Salzburg (AT)

Correspondence Address: Ladas & Parry, 26 West 61 Street, New York, NY, 10023, US

(73) Assignee: EMIL A.J. WIESER-LINHART

(21) Appl. No.: 09/860,033

(22) Filed: May 17, 2001

(30) Foreign Application Priority Data
Jul. 19, 2000 (CH) 1427/00

(51) Int. Cl. C12S 5/00; C12M 1/16

(52) U.S. Cl. 435/266; 435/299.1

(57) ABSTRACT

The biological washing reactor and the wet electrofilter are located in a common container. This container ends at the bottom at a biotank. A droplets remover is located at the purified gas outlet. The exhaust gas which is to be purified and which contains impurities in form of aerosols, dust and organic gases flows through the exhaust gas inlet into the container and flows firstly through the preliminary separator and thereafter through a biological washing reactor and the wet electrofilter. The process water for the continuous sprinkling of the preliminary separator and of the biological washing reactor, and for the intermittent cleaning of the wet electrofilter and of the droplets remover is circulated in a closed loop via the biotank. The circulated process water is regenerated in the biotank. The solid matter present in the portion of the process water which is fed to the biological washing reactor and to the droplets remover is separated and removed in a solids removing apparatus and this portion of the process water is cooled in a heat exchanger for an achieving of an optimal growth of bacteria.

### CROSS REFERENCE TO RELATED APPLICATIONS

This application claims the priority of Swiss patent application 1427/00, filed on Jul. 19, 2000, for which priority is claimed and the disclosure of which shall be considered as disclosed herein by reference thereto.

### BACKGROUND OF THE INVENTION

1. Field of the Invention

The present invention relates to a method of purifying an exhaust gas which includes impurities in form of aerosols, dust and organic gases. It also relates to an apparatus for purifying an exhaust gas which includes impurities in form of aerosols, dust and organic gases for practicing above method.

2. Description of the Related Art

Gases as described above are produced in a great many of industrial thermal conversion processes, such as typically at a mixed combustion, a hot compressing or also a thermal drying. As not limiting example wood pressing plants for the production of for instance chipboards can be mentioned.

The impurities to which reference is made herein are among others aerosols with a typical particle size of 0.1 μm. Such aerosols occur as so-called blue smoke. Further impurities encompass mists of liquids which are paraffin and oil mists with a typical particle size of 1 μm. These liquid mists occur as so-called white smoke. Still further impurities or contaminants, respectively, occur as flue dust with a typical particle size of 1-3 μm.

The fine particles are quite often nonhygroscopic, that is water repellent and are highly electrically loaded and, therefore, can only be separated and removed in wet elecrofilters. Conversely, organic gases can not be separated and removed in wet electrofilters.

### BRIEF SUMMARY OF THE INVENTION

Hence, it is a general object of the present invention to provide a method of purifying an exhaust gas by means of which fine particles and also organic gases can be simultaneously completely and perfectly separated and removed at low costs in an apparatus of a simple and compact design.

A further general object of the present invention is to provide an apparatus for purifying an exhaust gas by means of which fine particles and also organic gases can be simultaneously completely and perfectly separated and removed at low costs in an apparatus of a simple and compact design.

Still a further object of the invention is to provide a method wherein a separation of the impurities present in the exhaust gas is realized within one single operating cycle by a biological washing reactor and a wet electrofilter, onto which biological washing reactor process water is continuously sprinkled, and which wet electrofilter is intermittently cleaned by process water, which process water which is fed to the biological washing reactor and to the wet electrofilter is circulated in a closed loop which includes a biotank.

Yet a further object of the invention is to provide an apparatus which includes a container, a biological washing reactor and a wet electrofilter, which biological washing reactor and which wet electrofilter are both located in mentioned container.

The advantages gained by the invention may be seen specifically in that two different purifying procedures are united in one single cycle in a compact designed container, so that the purifying of the exhaust gas may be executed at low costs in one single cycle. For a treating of all operating units, may these be acted upon by process water continuously or intermitted for various reasons, only one single water circuit is needed.

### BRIEF DESCRIPTION OF THE DRAWINGS

The invention will be better understood and objects other than those set forth above, will become apparent when consideration is given to the following detailed description thereof. Such description makes reference to the annexed drawings, wherein:

FIG. 1 illustrates schematically a first embodiment of an apparatus for practicing the method according to the present invention; and

FIG. 2 illustrates schematically a second embodiment of an apparatus for practicing the method according to the present invention.

### DETAILED DESCRIPTION OF THE INVENTION

A biotank 2 which contains process water which is circulated in a closed loop is located at the lower portion of a container 1. The biotank 2 is equipped with a bottom aerator 3 which communicates with a source 4 of fresh air.

The biotank 2 communicates, furthermore, with a source 5 of a nutrient which will be described further below.

The exhaust gas to be purified enters the container 1 of the exhaust gas inlet 6.

This exhaust gas inlet 6 is located at a preliminary separator 7 which also functions to ensure an even distribution of the exhaust gas over the cross-section of the container 1. A sprinkling apparatus 8 is located above the preliminary separator 7. This sprinkling apparatus 8 communicates through a branch conduit 9 with an outflow conduit 10 of the biotank 2.

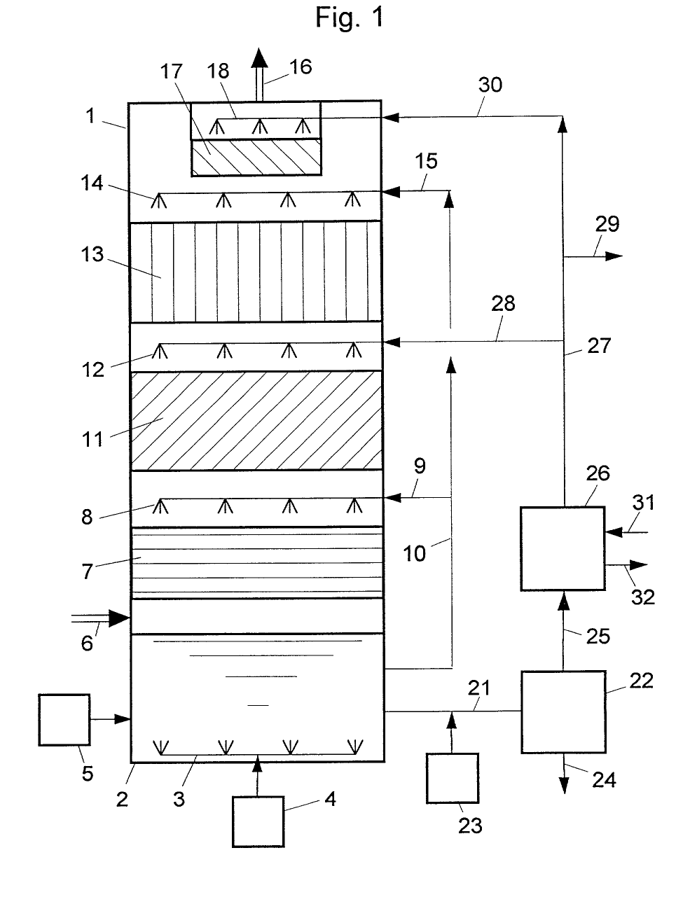

At the embodiment according to FIG. 1 a biological washing reactor 11 with a sprinkling apparatus 12 is located above the preliminary separator 7.

The feeding of the process water to the sprinkling apparatus 12 will be described further below.

A wet electrofilter 13 with a sprinkling apparatus 14 is located above the biological-washing reactor 11. This sprinkling apparatus 14 communicates through its feeding conduit 15 with the outflow conduit 10 of the biotank 2.

A droplets remover 17 is located at the purified gas outlet 16 on top of the container 1, and a sprinkling apparatus 18 is located above the droplets remover 17. The feeding of process water to this sprinkling apparatus 18 will also be described further below.

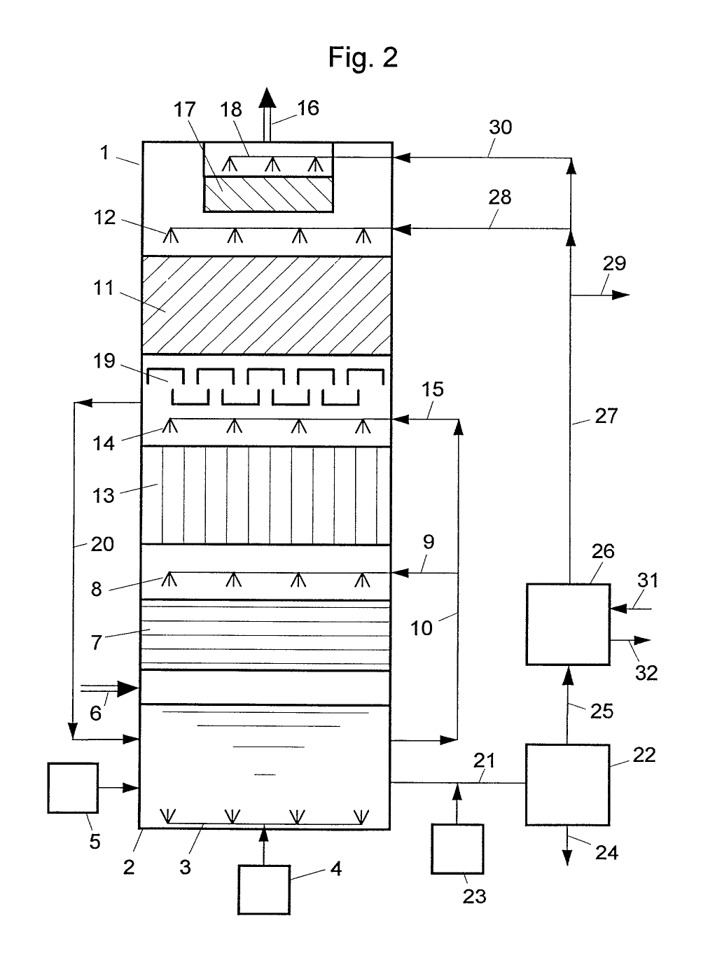

The design of the embodiment illustrated in FIG. 2 differs insofar from the design illustrated in FIG. 1 in that the biological washing reactor 11 is located above the wet electrofilter 13. This embodiment includes a partition wall 19 which is inserted between the biological washing reactor 11 and the wet electrofilter 13. An outflow conduit 20 runs directly from this partition wall 19 to the biotank 2, which outflow conduit 20 circumnavigates, runs around the wet electrofilter 13.

A further outflow conduit 21 extends at both embodiments from the biotank 2 to a solids removing apparatus 22. This outflow line 21 communicates with a source 23 of a flocculation agent. The reference numeral 24 designates the draining line for the separated solid matter.

A conduit 25 extends from the solids removing apparatus 22 to a heat exchanger 26.

At the embodiment according to FIG. 1 the feeding conduit 28 running to the sprinkling apparatus 12 of the biological washing reactor 11 is branched off the outflow conduit 27 of the heat exchanger 26.

The reference numeral 29 designates an outlet conduit for a draining off of superfluous water.

Finally, the outflow conduit 27 runs to the feeding conduit 30 of the sprinkling apparatus 18 of the droplet remover 17.

At the embodiment according to FIG. 2, the feeding conduit 28 for the sprinkling apparatus 12 of the biological washing reactor 11 branches off not earlier than after the outlet conduit 29. Also illustrated is again the feeding conduit 30 to the sprinkling apparatus 18 of the droplets remover 17.

As generally can be seen, the disclosed alternatives differ from each other substantially in that according to the embodiment of FIG. 1 the biological washing reactor 11 is located under the wet electrofilter 13, and according to the embodiment of FIG. 2 the wet electrofilter 13 is located under the biological washing reactor 11.

At both embodiments the exhaust gas to be purified enters the container 1 of the exhaust gas inlet 6 and is fed through the preliminary separator 7. Process water is continuously sprinkled over this preliminary separator, which process water is fed to the sprinkling apparatus 8 from the biotank 2 through the conduits 9 and 10 and drops in a closed circuit or loop, respectively, back into the biotank 2. In the preliminary separator 7 there occurs at the one hand the distribution of the gas over the cross-section of the container 1, but at the other hand, such as will be describe further below, a cooling of the exhaust gas and a preliminary separating and removing of coarse constituents of the exhaust gas.

At the embodiment according to FIG. 1 the exhaust gas, after leaving the preliminary separator 7, enters the biological washing reactor 11 which is filled by a structured biomass.

At the embodiment according to FIG. 1 the exhaust gas, after leaving the preliminary separator 7, enters a honeycomb-like designed wet electrofilter 13.

The flow of the gas proceeds in the biological washing reactor 11 and in the wet electrofilter upwards from the bottom to the top.

In the embodiment according to FIG. 2 the wet electrofilter 13 is protected by the partition wall 19 from the sprinkled water dropping off the biological washing reactor 11, which sprinkling water is returned directly to the biotank 2 through the outflow conduit 20.

The wet electrofilter 13 is cleaned intermittently by circulating water which is fed untreated from the biotank 2 through the conduits 10 and 15 to the sprinkling apparatus 14.

The biological washing reactor 11 is continuously sprinkled by pre-treated process water which flows from the biotank 2 through the outflow conduit 21 to the solids removing apparatus 22, thereafter through the heat exchanger 26 and finally through the conduits 27 and 28 to the sprinkling apparatus 12. The film of bacteria which has grown in the biological-washing reactor 11 absorbs the organic gases and oxidizes these gases.

The purified exhaust gas flows finally out of the container 12 through the purified gas outlet 16 and flows beforehand through the droplets remover 17 which is also intermittently cleaned by process water which has been pre-treated in the solids removing apparatus 22 and the heat exchanger 26. This process water flows from the heat exchanger 26 through the conduits 27 and 30 to the sprinkling apparatus 18 which is located above the droplets remover 17.

Fresh air is fed from the source 4 for fresh air into the biotank 4. Nutrients for the supply of nitrogen and phosphorus are fed from the source of nutrients 5 into the biotank 4.

Polyelectrolyte acting as flocculation agent is added from the flocculation agent source 23 to the process water which flows to the solids removing apparatus 22. The solid matter which has been separated in the solids removing apparatus 22 is drained off or discharged, respectively, through the draining line 24.

Excess waste water which stems from the partial condensation of the humidity of the raw gas, that is the humidity of the fed exhaust gas to be purified, is drained off the circuit through the outlet conduit 29 which branches off from the outflow conduit 27.

The selection between the embodiments according to FIG. 1 and FIG. 2 depends from the composition of the exhaust gas which is to be purified.

When the exhaust gas which is to be purified has a low content of aerosols, the arrangement of the wet electrofilter 13 over the biological washing reactor 11 according to FIG. 1 is selected, in which embodiment there is no partition wall 19, so that the apparatus is of a somewhat more simple design.

In case of a high content of aerosols, the arrangement according to FIG. 2 is to be selected, because due to this design an additional contamination of the biological washing reactor 11 by the cleaning water coming from the wet electrofilter 13 is avoided.

It is to be noted that the apparatus is designed in such a manner, that the speed of flow of the exhaust gas amounts in both the wet electrofilter 13 and the biological washing reactor 11 11-13 meters per second, and that the dwell time in both the wet electrofilter 13 and the biological washing reactor 11 amounts to 1-16 seconds.

Below, the individual steps of the treatment of the exhaust gas will now be described.

The exhaust gas which enters the apparatus through the exhaust gas inlet 6 has a temperature in the range of 60°-200° C. This exhaust gas is initially cooled by the sprinkling water flowing out of the preliminary separator 7 by a evaporative cooling down to the primary point of condensation amounting to 50°-70° C.

The exhaust gas flows thereafter through the preliminary separator 7 which is composed of trickling plates and causes additionally the distribution of the gas over the cross-section of the container 1 and accordingly the biological washing reactor 11 and the wet electrofilter 13. The preliminary separator allows the passage of coarse, wettable particles with a size >10 μm and also the absorption of easily water-soluble gas constituents. The process, that is sprinkling water which is fed through the sprinkling apparatus 8 to the preliminary separator 7 has previously been regenerated in the biotank 2 and can accordingly absorb impurities.

The exhaust gas flows thereafter through the biological washing reactor 11 followed by the wet electrofilter 13 according to the embodiment of FIG. 1, or through the wet electrofilter 13 followed by the biological washing reactor 11 according to the embodiment of FIG. 2.

The biological washing reactor 11 is composed of structured growth elements (packing material) which allow at the one hand a growth of the biological mass and at the other hand a easy rinsing of the grown film. The biological washing reactor 11 is continuously sprinkled upon. The absorption of the organic gases and the bio-catalytic oxidation proceed by the film of water present and the layer of bacteriae located under this film. The bacteriae is accordingly supplied from the gas with substrate and from the process water, that is the sprinkled water.

This process water is supplied from the biotank 2. The process water in the biotank 2 is supplied from the source 5 with nutrients for the bacteriae, such as typically urea and phosphoric acid for the supply of Nitrogen N and phosphorous P. In the solids removing apparatus 22 solid water is separated and removed from the process water which has been drawn off the biotank 2 and to which a flocculation agent for supporting the flocculation has been added from the source 23. The flocculation agent is obviously added for a improving of the separating capacity of the solids removing apparatus 22, because the constituents of the process water which flows out of the biotank can be fine disperse. Depending from the prevailing constituents the solids removing apparatus 22 can be designed as a filter, a flotation apparatus or a sedimentation apparatus. In the heat exchanger 26 which follows the solids removing apparatus 22 the process water, which is now a clear water, is cooled 5°-20° C. down to a temperature range of 35°-55° C. This cooling causes a further lowering of the primary dew point, which allows at the one hand the maintaining of the optimal temperature range of 35°-55° C. for the growth of the bacteriae, and produces by condensation waste water which is drawn off through the outlet conduit 29.

Accordingly, the process temperature can be optimized and held at a constant value depending from the composition of the gas and the humidity and the necessary degree of the decomposition.

The cooling water supply for the heat exchanger 26, see connections 31, 32 may come from a closed cooling tower loop, from ground or surface water or also from a heat pump which feeds the waste heat into a district heating network.

By the process water which is treated as set forth above and which is sprinkled over the biological washing reactor optimal conditions for the disintegration of the organic constituents of the exhaust gas which is to be purified are reached. The biological mass optimized itself from the mixed population on its own. When starting the apparatus up a balanced state between the growing and the rinsed off mass of the bacteriae is produced, that is a newly installed biological washing reactor is completely operative after about 4 weeks.

The wet electrofilter 13 which is located either above or below the biological washing reactor 11 is composed of hexagonal, tube-shaped honeycomb elements and has a typical width over flats of the hexagon of 200-400 mm of a length of 2-6 meters. A electrical discharge wire, which is mounted to a upper and a lower mounting frame, is located at the center of each tube which forms a single honeycomb element. The upper mounting frame is held in insulating chambers and is supplied externally with a negative direct voltage of 50-110 kV. The voltage and the current are controlled automatically so that minor or no flashovers occur. The negative corona at the electrical discharge wire causes an electric wind towards the hexagonal inner surface of the tube which operates as a precipitation electrode and the emitted electrons ionize the constituents of the exhaust gas, predominantly the aerosols by a ionization by impact. When now the fine particles are loaded electrically, they move corresponding to the direction of the electrical wind perpendicularly to the direction of flow of the gas in the corresponding tube onto the inner wall of the tube were they a separated out as a coating, are deposited and accordingly are removed out of the gas flow.

This coating is intermittently rinsed off to flow downwards by means of circulated process water and enters, finally, the biotank 2.

The partition wall 19 which in the embodiment of FIG. 2 is located above the wet electrofilter 13 prevents the process water which is dripping off the biological washing reactor 11 to cause a voltage drop in the wet electrofilter 13.

A droplets remover 17 is located ahead of the purified gas outlet 16, that is the chimney, which droplet remover 17 prevents droplet which are entrained in the gas exiting the biological washing reactor 11 or the wet electrofilter 13 from flowing into the chimney. This droplets remover 17 is intermittently rinsed automatically by purified process water.

The process water which is circulated in a loop and which flows continuously specifically from the biological washing reactor 11 and from the preliminary separator 7 into the biotank 2, and which flows intermittently from the wet electrofilter 13 and from the droplets remover 17 into the biotank 2 is collected in the biotank 2. Therefore, the biotank 2 contains all solid matter which has been separated and removed and contains activated sludge and is, such as has been already mentioned, aerated from the source 4 of fresh air through the bottom aerator 3. Correspondingly, the further oxidation of the organic constituents of the exhaust gas takes place in the biotank 2.

While there are shown and described present preferred embodiments of the invention, it is to be distinctly understood that the invention is not limited thereto, but may be otherwise variously embodied and practiced within the scope of the following claims.

1. A method of purifying an exhaust gas which includes impurities in form of aerosoles, dust and organic cases, wherein a separation of the impurities present in the exhaust gas is realized within one single operating cycle by a biological washing reactor and a wet electrofilter, onto which biological washing reactor process water is continuously sprinkled, and which wet electrofilter is intermittently cleaned by process water, which process water which is fed to the biological washing reactor and to the wet electrofilter is circulated in a closed loop which includes a biotank.

2. The method of claim 1, wherein process water which has been regenerated in the biotank, cooled in a heat exchanger and cleaned in a solid separating apparatus is fed to the biological washing reactor onto which process water is continuously sprinkled, and wherein the process water by means of which the wet electrofilter is intermittently cleaned is directly drawn out of the biotank.

3. The method of claim 2, wherein the exhaust gas, prior to flowing through the biological washing reactor and through to wet electrofilter, is fed through a preliminary separator, which preliminary separator operates simultaneously as gas distributor and wherein process water which is drawn out of the biotank is continuously sprinkled onto the preliminary separator.

4. The method of claim 1, wherein exhaust gas which has been purified in the biological washing reactor and in the wet electrofilter is subjected to a removal of droplets in a droplets removing apparatus, which droplet removing apparatus is intermittently cleaned by process water which has been cooled in a heat exchanger and cleaned in a solids separating apparatus.

5. The method of claim 1, wherein the operating temperature of the process water which is circulated in a closed loop is held constant at varying humidities of the exhaust gas by a controlled removing of heat in a heat exchanger.

6. The method of claim 3, wherein the exhaust gas which has passed through the preliminary separator is led firstly through the wet electrofilter and thereafter through the biological washing reactor.

7. The method of claim 3, wherein the biological washing reactor is located above the wet electrofilter, and wherein the process water which has been sprinkled in the biological washing reactor and flows out of same is directly fed to the biotank circumventing the wet electrofilter.

8. The method of claim 3, wherein the exhaust gas which has passed through the preliminary separator is led firstly through the biological washing reactor and thereafter through the wet electrofilter.

9. The method of claim 1, wherein the speed of passage of the exhaust gas being purified amounts to 1-3 meters per second in the biological washing reactor and also to 1-3 meters per second in the wet electrofilter, and wherein the dwell time of the exhaust gas being purified amounts to 1-6 seconds in the biological washing reactor and to 1-6 seconds in the wet electrofilter.

10. An apparatus for purifying an exhaust gas which includes impurities in form of aerosols, dust and organic gases, said apparatus comprising a container, a biological washing reactor and a wet electrofilter, which biological washing reactor and which wet electrofilter are both located in said container.

11. The apparatus of claim 10, comprising a sprinkling apparatus adapted for a continuous sprinkling of the biological washing reactor, and comprising a further sprinkling apparatus adapted for a intermittent cleaning of the wet electrofilter, further comprising a biotank located under the biological washing reactor and the wet electrofilter, which biotank communicates through conduits for a circulating of process water in a closed loop with the sprinkling apparatus adapted for a continuous sprinkling the biological washing reactor, and with further sprinkling apparatus adapted for a intermittent cleaning of the wet electrofilter.

12. The apparatus of claim 10, wherein the container comprises an inlet for the exhaust gas to be purified, which exhaust gas inlet is located below the biological washing reactor, below the wet electrofilter and above the biotank.

13. The apparatus of claim 11, comprising a preliminary separator which is located at the exhaust gas inlet, which preliminary separator is adapted to ensure an even gas distribution in the container; and comprising a sprinkling apparatus located above the preliminary separator and supplied with a portion of the process water, which sprinkling apparatus communicates through an arrangement of conduits with the biotank.

14. The apparatus of claim 11, wherein the sprinkling apparatus adapted for a intermittent cleaning of the wet electrofilter is supplied with a flow of a part of the process water and communicates through an arrangement of conduits directly with the biotank.

15. The apparatus of claim 11, comprising a solids separator apparatus and a heat exchanger; a conduit for a flow of a part of the process water extending from the biotank to the solids separator apparatus; which solids separator apparatus is followed by the heat exchanger for a cooling of this part of the process water; which heat exchanger communicates through an arrangement of conduits with the sprinkling apparatus adapted for a continuous sprinkling of the biological washing reactor.

16. The apparatus of claim 15, wherein the container comprises a purified gas outlet and a droplets removing apparatus located at the purified gas outlet; and comprises a sprinkling apparatus adapted for an intermittent cleaning of the droplets removing apparatus located above the droplet removing apparatus; which sprinkling apparatus communicates with the heat exchanger so to be supplied with cleaned, cooled process water.

17. The apparatus of claim 11, wherein the biotank communicates with a source for a feeding of a nutrient for a supply with nitrogen/phosphorous thereto, and with a further source for a feeding of fresh air thereto.

18. The apparatus of claim 15, wherein the conduit for a flow of a part of the process water extending from the biotank to the solids separator apparatus communicates with a source for a feeding of a flocculation agent thereto.

19. The apparatus of claim 15, comprising an outlet conduit located regarding the process water downstream of the heat exchanger for a removing of superfluous water.

20. The apparatus of claim 11, wherein the wet electrofilter is located in the container above the biological washing reactor, so that the process water which intermittently drops off the wet electrofilter flows through the biological washing reactor.

21. The apparatus of claim 11, wherein the biological washing reactor is located in the container above the wet electrofilter and a partition wall for receiving the process water which continuously drips off the biological washing reactor, which partition wall communicates through a discharging conduit which circumnavigates the wet electrofilter directly with the biotank.
